(19) United States
(12) Patent Application Publication
(10) Pub. No.: US 2019/0358021 A1
(43) Pub. Date: Nov. 28, 2019

(54) MEDICAL MEMBER AND METHOD FOR TREATING SOFT TISSUE

(71) Applicant: NIPPON PISTON RING CO., LTD., Saitama City, Saitama (JP)

(72) Inventors: Takasumi KUBO, Saitama (JP); Yoshiki ISHIKAWA, Saitama (JP); Hiroshi MATSUSHIMA, Saitama (JP); Yuki KIMURA, Saitama (JP); Masahiro SHINZAWA, Saitama City, Saitama (JP); Takashi KAWABATA, Hasuda (JP); Toshio NAGASE, Tsukuba (JP)

(73) Assignees: NIPPON PISTON RING CO., LTD., Saitama City, Saitama (JP); NIPPON PISTON RING CO., LTD., Saitama City, Saitama (JP)

(21) Appl. No.: 16/479,554

(22) PCT Filed: Jan. 18, 2018
(86) PCT No.: PCT/JP2018/001349

§ 371 Date: Jul. 19, 2019

(30) Foreign Application Priority Data
Jan. 20, 2017 (JP) 2017-008116

(51) Int. Cl. A61F 2/08; A61B 17/04; A61B 17/12; A61F 2/07; A61L 27/06

(52) U.S. Cl. CPC A61F 2/08; A61B 17/0401; A61F 2002/823; A61F 2/07; A61L 27/06; A61B 17/12022

(57) ABSTRACT

A medical member of the present invention includes a knitted body knitted with a linear member formed in a linear shape, at least a part of the knitted body is constituted of an alloy containing titanium, tantalum, and tin, and the alloy is constituted of 15 at % to 27 at % of tantalum and 1 at % to 8 at % of tin, where the total is 100 at %, with remainder being titanium and unavoidable impurities. The knitted body is formed in a sheet shape by plain weaving, twilled weaving, plain dutch weaving, twilled dutch weaving, or stockinette stitch. Thus, there can be provided a medical member capable of dispersing a load applied to a treating site of a soft tissue.

### CROSS-REFERENCE TO RELATED APPLICATIONS

This application is a U.S. National Phase Application of International Application No. PCT/JP2018/001349, filed on Jan. 18, 2018, which claims priority to Japanese Application No. 2017-008116, filed on Jan. 20, 2017. The entire disclosures of the above applications are incorporated herein by reference.

### BACKGROUND

### Technical Field

The present invention relates to a medical member and a method for treating a soft tissue.

### Related Art

A torn soft tissue is conventionally treated by passing a suture thread through the soft tissue to close the torn site and then tying the suture thread (for example, see Japanese Patent Application Laid-Open No. 2011-025036). Further, as an instrument for treating the torn soft tissue, there is proposed a surgical assembly including a fastener in which a male screw is formed, a retainer, a suture thread, and a slip knot (for example, see WO2004/037094). The fastener and the retainer are coupled together by the suture thread. The suture thread is tied in the slip knot such that the distance between the fastener and the retainer is shortened by pulling a free end of the suture thread.

This surgical assembly is used, for example, for treating a damage in the rotator cuff. In the treatment, first, the fastener is passed through the rotator cuff tissue and inserted into the humerus near the rotator cuff. Then, pulling the free end of the suture thread allows the retainer and the slip knot to move toward the fastener. As a result, the rotator cuff tissue is pressed against the humerus by the retainer and the slip knot. In this manner, the rotator cuff is fixed to the humerus.

Further, in recent years, a nickel-titanium alloy is often used as a material of an instrument indwelled inside the living body (hereinafter, referred to as “in-body indwelling instrument”) such as the fastener described above. Under such circumstances, the applicant of the present application proposes, as a material compatible with the living body, a titanium alloy containing tantalum (Ta) and tin (Sn) with the remainder being titanium (Ti) and unavoidable impurities (for example, see WO2012/105557).

Further, in treating the human body, a stent is used when a tubular part of the human body such as a blood vessel needs to be expanded from its inside. As the stent, for example, an instrument having a long narrow wire knitted into a tubular structure has been proposed (for example, see WO2006/124541). The publication discloses that the wire is preferably formed of nitinol. Further, the nitinol is generally constituted of a nickel-titanium alloy.

When the torn site of the soft tissue is closed by the suture thread in the treatment, a load applied to the suture site is concentrated on the suture thread and the soft tissue around the suture thread. Further, when the damage in the rotator cuff is treated using the surgical assembly described above, a load applied to the fixed site of the rotator cuff is concentrated on the suture thread and the soft tissue around the suture thread. The load concentrated on the suture thread and the soft tissue around the suture thread described above may cause a break in the suture thread or reopen the torn site.

Further, nickel is a substance which may cause an allergy to the living body. Thus, it is preferable to form the in-body indwelling instrument using a material free from nickel from the standpoint of ensuring the safety of the living body.

Further, the nickel-titanium alloy has strong magnetic properties. Thus, when a diagnosis using MRI is performed in a state in which the in-body indwelling instrument is indwelled in the living body, there may arise a problem in that the in-body indwelling instrument is heated by a magnetic field generated by the MRI equipment.

Further, the nickel-titanium alloy exhibits a low X-ray absorbance. Thus, when a location of the in-body indwelling instrument is confirmed using an X-ray photographic image at the time of the follow-up observation of the tissue treatment, it sometimes takes time to locate the in-body indwelling instrument.

The present invention has been made in view of the above situation, and an object of the present invention is to provide a medical member capable of dispersing a load applied to a treating site of a soft tissue and a method for treating a soft tissue.

### SUMMARY OF THE INVENTION

The present invention has been made to solve the above-mentioned problems, and the medical member of the present invention includes a knitted body knitted with a linear member formed in a linear shape, at least a part of the knitted body is constituted of an alloy containing titanium, tantalum, and tin, and the alloy is constituted of 15 at % to 27 at % of tantalum and 1 at % to 8 at % of tin, where the total is 100 at %, with remainder being titanium and unavoidable impurities.

Further, in the medical member of the present invention, the knitted body is formed in a sheet shape.

Further, in the medical member of the present invention, the knitted body is formed by plain weaving, twilled weaving, plain dutch weaving, twilled dutch weaving, or stockinette stitch.

Further, in the medical member of the present invention, the knitted body is constituted by a plurality of knitted body elements, and the knitted body elements are knitted with the linear members in respective different modes and thus have respective different stretching rates, and the stretching rate is represented by a strain per unit tensile force in a tensile direction of the tensile force.

Further, in the medical member of the present invention, the plurality of knitted body elements are knitted such that the stretching rate of a predetermined one of the knitted body elements is limited by at least one of the remaining knitted body elements knitted into the predetermined knitted body element.

Further, in the medical member of the present invention, the knitted body is configured to be attachable to a soft tissue failure site, the soft tissue failure site being a torn site, a damage site, or a defect site of the soft tissue.

Further, in the medical member of the present invention, the knitted body includes a first region portion configured to cover a treating region where the soft tissue failure site is treated and a second region portion configured to cover a peripheral region of the treating region and be allowed to engage with the soft tissue.

Further, the medical member of the present invention includes an engaging member configured to engage the knitted body with the soft tissue.

Further, in the medical member of the present invention, the engaging member is a thread member capable of stitching the soft tissue and the knitted body together.

Further, in the medical member of the present invention, the knitted body is constituted by a net-shaped main body and a passage penetrating the main body.

Further, in the medical member of the present invention, the knitted body is a stent.

Further, the medical member of the present invention is constituted by a stent graft configured to include the stent inside an artificial blood vessel.

Further, in the medical member of the present invention, the knitted body is an embolization member configured to block a blood flow in a blood vessel.

Further, in the medical member of the present invention, the knitted body has stretchability.

Further, in the medical member of the present invention, the knitted body has a stretching rate of 120% or more, the stretching rate being represented by a strain per unit tensile force in a tensile direction of the tensile force.

Further, the medical member of the present invention includes a non-integrated surface that is formed of a non-integrated biomaterial having a property of not being integrated with a biotissue.

Further, in the medical member of the present invention, a surface of the knitted body is disposed on a surface opposite to the non-integrated surface.

Further, the medical member of the present invention includes a non-integrated biomaterial having the property of not being integrated with a biotissue, and the non-integrated biomaterial is disposed on the knitted body to form a layer.

Further, in the medical member of the present invention, the non-integrated biomaterial is a polymer containing at least one component of fluoroethylene, fluoropropylene, and fluoro-polyethylene glycol, a non-crosslinkable silicone polymer, or 2-methacryloyloxyethyl phosphorylcholine.

Further, the medical member of the present invention includes a polymer portion formed of a polymer.

Further, in the medical member of the present invention, the polymer portion is formed as a layer of the polymer configured to coat the surface of the knitted body.

Further, in the medical member of the present invention, the polymer portion is formed in a net shape or a sheet shape and consecutively connected to the knitted body.

Further, the medical member of the present invention includes a layer of hydroxyapatite configured to coat the knitted body.

Further, in the medical member of the present invention, a method for treating a soft tissue using any of the medical members described above includes attaching the knitted body to a soft tissue failure site.

Further, in the method for treating the soft tissue of the present invention, the treating region is covered with the knitted body and the knitted body is engaged with the soft tissue at a peripheral region of the treating region.

Further, in the method for treating the soft tissue of the present invention, the knitted body is engaged with the soft tissue by stitching.

Further, the method for treating a soft tissue of the present invention is a method using the knitted body (the medical member) constituted by the net-shaped main body and the passage penetrating the main body, the knitted body being attached to the soft tissue failure site by inserting the soft tissue failure site in the passage of the knitted body.

Further, in the method for treating the soft tissue of the present invention, the soft tissue failure site to which the knitted body is to be attached is fixed to any of living tissues by a fixing member, the fixing member includes a pair of engaging members configured to be engaged with the living tissues and a thread member configured to be connected to the pair of engaging members, and the fixing member is disposed such that the knitted body is pressed against the living tissue by the thread member.

### Advantageous Effects of the Invention

The medical member and the method for treating the soft tissue of the present invention can exhibit the excellent effect of dispersing the load applied to the treating site of a soft tissue.

### BRIEF DESCRIPTION OF THE DRAWINGS

FIG. 1(A) is a perspective view illustrating a medical member in a first embodiment of the present invention. FIG. 1(B) is a plan view illustrating a net-shaped body in the first embodiment of the present invention.

FIGS. 2(A) to 2(D) are diagrams each illustrating a procedure for treating a tear of a soft tissue using the medical member in the first embodiment of the present invention, arranged in time series from (A) to (D).

FIG. 3(A) is a diagram illustrating an example of a rotator cuff tear. FIG. 3(B) is a diagram illustrating how the rotator cuff tear is treated using the medical member in the first embodiment of the present invention.

FIG. 4(A) is a diagram illustrating another example of the rotator cuff tear. FIG. 4(B) is a diagram illustrating how the rotator cuff tear is treated using the medical member in the first embodiment of the present invention.

FIG. 5 is a plan view illustrating the net-shaped body in the first embodiment of the present invention.

FIG. 6 is a perspective view illustrating a medical member in a second embodiment of the present invention.

FIG. 7(A) is a perspective view illustrating a medical member in a third embodiment of the present invention. FIG. 7(B) is a perspective view illustrating the medical member in the third embodiment formed by using the medical member in the first embodiment of the present invention.

FIGS. 8(A) to 8(C) are diagrams each illustrating the procedure for treating the tear of the soft tissue using the medical member in the third embodiment of the present invention, arranged in time series from (A) to (C).

FIG. 9 is a perspective view illustrating a medical member in a fourth embodiment of the present invention.

FIG. 10(A) is a cross-sectional view illustrating a layer body constituting a medical member in a fifth embodiment of the present invention and a perspective view illustrating a net-shaped body and a sheet body constituting the layer body. FIG. 10(B) is a perspective view illustrating a modification of the layer body constituting the medical member in the fifth embodiment of the present invention.

FIG. 11(A) is a diagram illustrating an example of a hernia. FIG. 11(B) is a diagram illustrating how the hernia is treated using the layer body constituting the medical member in the fifth embodiment of the present invention. FIG. 11(C) is a diagram illustrating how the hernia is treated using the modification of the layer body constituting the medical member in the fifth embodiment of the present invention.

FIG. 12(A) is a cross-sectional view illustrating the layer body constituting the medical member in the fifth embodiment of the present invention. FIG. 12(B) is a perspective view illustrating a projecting body constituting the medical member in the fifth embodiment of the present invention. FIG. 12(C) is a perspective view illustrating a modification of the projecting body constituting the medical member in the fifth embodiment of the present invention.

FIG. 13(A) is a diagram illustrating how the hernia is treated using the projecting body constituting the medical member in the fifth embodiment of the present invention. FIG. 13(B) is a diagram illustrating how the hernia is treated using the modification of the projecting body constituting the medical member in the fifth embodiment of the present invention.

FIG. 14(A) is a cross-sectional view illustrating a first modification of the layer body constituting the medical member in the fifth embodiment of the present invention. FIG. 14(B) is a cross-sectional view illustrating a second modification of the layer body constituting the medical member in the fifth embodiment of the present invention.

FIG. 15(A) is a perspective view illustrating a medical member in a sixth embodiment of the present invention. FIG. 15(B) is a perspective view illustrating a medical member in a seventh embodiment of the present invention.

FIG. 16 is a perspective view illustrating a medical member in an eighth embodiment of the present invention.

FIG. 17(A) is a plan view illustrating a medical member in a ninth embodiment of the present invention. FIGS. 17(B) and 17(C) are plan views each illustrating a knitted body element constituting the medical member in the ninth embodiment of the present invention.

FIG. 18(A) is a plan view illustrating a medical member in a tenth embodiment of the present invention. FIGS. 18(B) and 18(C) are plan views each illustrating a knitted body element constituting the medical member in the tenth embodiment of the present invention.

### DETAILED DESCRIPTION OF THE PREFERRED EMBODIMENTS

Embodiments of the present invention will next be described with reference to the attached drawings. Note that some parts of the drawings are omitted or simplified in order to facilitate understanding of the drawings. Further, shapes and dimensional ratios of portions in the respective drawings are not necessarily exactly depicted.

### 1. First Embodiment

### 1-1. Overall Configuration

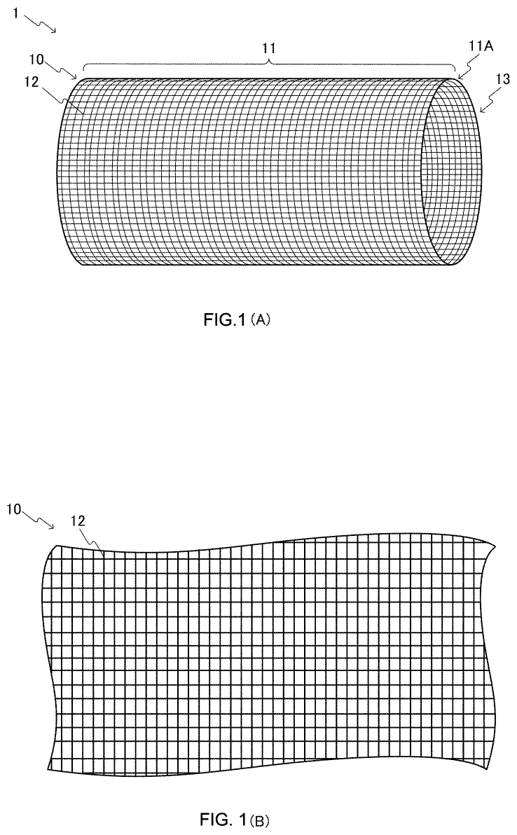

Referring to FIG. 1, a medical member 1 in the first embodiment of the present invention will be described below. The medical member 1 is attached to a soft tissue failure site, which is a tear site, a damage site, or a defect site of a soft tissue. As shown in FIG. 1(A), the medical member 1 is constituted by a net-shaped cylindrical body 11 in which a net-shaped body 10 is formed in a cylindrical shape. Further, the net-shaped cylindrical body 11 is constituted by a net-shaped main body 11A and a passage 13 penetrating the main body. The soft tissue failure site is inserted in the passage 13.

The net-shaped body 10 is a net-shaped sheet body. Specifically, the net-shaped body 10 is, for examples, as shown in FIG. 1(B), constituted by a knitted body knitted with a linear member 12 formed in a linear shape. Further, the knitted body is knitted with the linear member 12 in a sheet shape. The linear member 12 may be, for example, any of a round wire having a substantially circular section or a substantially elliptical section, a flat wire or ribbon wire having a substantially rectangular section, and a wire having a section of any other shape (for example, a polygon other than rectangle). Further, the diameter of the section of the linear member 12 may be of various sizes, but, for example, it is preferably about 10 to 60 μm.

The knitted body can be formed by a method of, for example, plain weaving, twilled weaving, plain dutch weaving, twilled dutch weaving, or stockinette stitch. Further, the knitted body is not necessarily formed in the sheet shape and, for example, it may be three-dimensionally knitted by the stockinette stitch. Further, the knitted body may be formed in the sheet shape or three-dimensionally using the linear member 12 by a method other than the methods described above.

The net-shaped body 10 (the knitted body) or the net-shaped cylindrical body 11 preferably has a stretching rate of, for example, 120% or more. The net-shaped body 10 or the net-shaped cylindrical body 11 having the stretching rate of 120% or more is durable in use at any site. Note that the stretching rate refers to a strain per unit tensile force in a tensile direction of the tensile force. The strain is a ratio of stretching in the tensile direction relative to the original length. The same applies hereinafter.

The net-shaped body 10 (the knitted body) has biocompatibility and stretchability. Note that the stretching rate of the net-shaped body 10 (the knitted body) can be controlled by a mesh density, a mesh direction, and the like. Thus, it is preferable to change the mesh density, the mesh direction, and the like of the net-shaped body 10 (the knitted body) depending on a site where the net-shaped cylindrical body 11 is used. For example, it is preferable to use the net-shaped body 10 (the knitted body) knitted so as to reduce the stretching rate at a site where bending and stretching of the soft tissue is desired to be limited. On the other hand, it is preferable to use the net-shaped body 10 (the knitted body) knitted so as to increase the stretching rate at a site where bending and stretching of the soft tissue are tolerated. In this manner, the net-shaped body 10 (the knitted body) is easily deformable by following the bending and stretching of the soft tissue.

### 1-2. Method for Treating Soft Tissue Using Net-Shaped Cylindrical Body

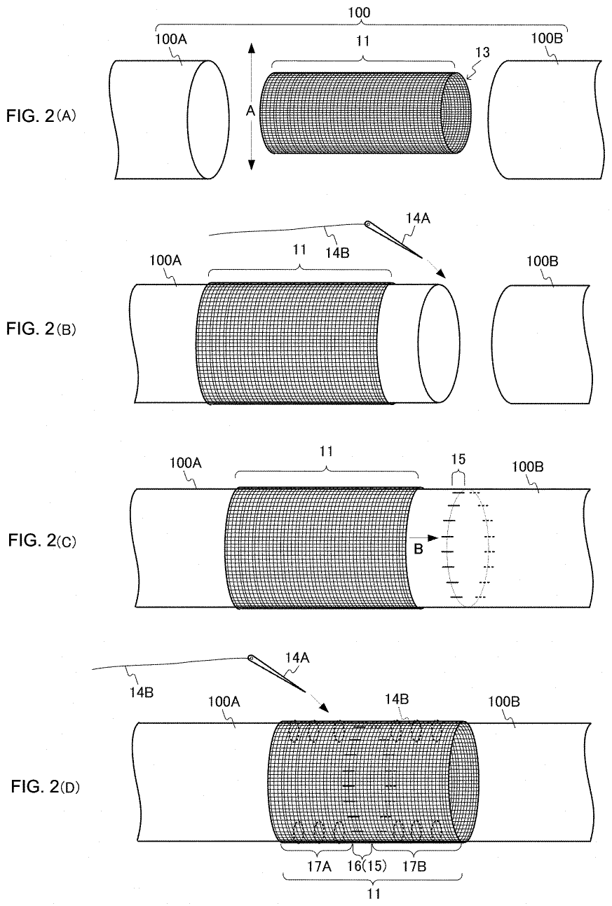

A using method of the net-shaped cylindrical body 11 will be described with reference to FIG. 2. The net-shaped cylindrical body 11 is, for example, attached to the soft tissue failure site. The soft tissue failure site refers to a tear site, a damage site, a defect site, or the like of the soft tissue, and the same applies hereinafter. Further, the soft tissue refers to a supporting tissue other than a skeleton in the living body. Specific examples of the soft tissue may include a connective tissue excluding a bone tissue such as a tendon, a ligament, a fascia, a skin, and a fat tissue, a blood vessel, a striated muscle, a smooth muscle, and a peripheral nervous tissue (a ganglion and a nerve fiber), or the like.

A description will next be given of a case where the net-shaped cylindrical body 11 is attached to disconnected soft tissues 100A and 100B caused by a tear of a soft tissue 100. First, as shown in FIG. 2(A), the diameter of the soft tissue 100A is larger than that of the net-shaped cylindrical body 11, and thus the diameter of the net-shaped cylindrical body 11 is expanded (see an arrow A direction). Then, as shown in FIG. 2(B), the soft tissue 100A, one of the torn tissues, is inserted in the passage 13 of the net-shaped cylindrical body 11 such that an outer circumferential surface of the soft tissue 100A is covered with the net-shaped cylindrical body 11.

Next, the disconnected soft tissue 100A and soft tissue 100B are stitched together using a needle member 14A and a thread member 14B. Upon completion of stitching, as shown in FIG. 2(C), the net-shaped cylindrical body 11 is slid along the soft tissue 100A (see an arrow B direction). In this operation, as shown in FIG. 2(D), the net-shaped cylindrical body 11 is slid so as to be disposed across a treating region 15. Note that the treating region 15 refers to a region occupied by a treating member (for example, the thread member 14B) at the torn site and a surrounding region thereof of the soft tissue 100 when the torn site of the soft tissue 100 is treated by stitching or the like. Thus, it can be said that the net-shaped cylindrical body 11 includes a first region 16 that covers the treating region 15 and second regions 17A and 17B that cover the surrounding regions of the treating region 15.

The second regions 17A and 17B of the net-shaped cylindrical body 11 are regions where the net-shaped cylindrical body 11 is allowed to engage with the soft tissues 100A and 100B, respectively. The engagement of the second regions 17A and 17B of the net-shaped cylindrical body 11 with the soft tissues 100A and 100B can be achieved, for example, as shown in FIG. 2(D), by stitching the net-shaped cylindrical body 11 and the soft tissues 100A and 100B together using the needle member 14A and the thread member 14B, without being limited thereto. The engagement of the net-shaped cylindrical body 11 with the soft tissues 100A and 100B may be achieved, for example, by other engaging members such as a skin stapler.

As described above, when the net-shaped cylindrical body 11 is attached to the torn site of the soft tissue 100, the load applied to the treating region 15 is dispersed to a surrounding area via the net-shaped cylindrical body 11. Further, the net-shaped cylindrical body 11 has stretchability. Thus, the net-shaped cylindrical body 11 allows the torn site of the soft tissue 100 to deform without imposing an excessive load on the torn site of the soft tissue 100. In this manner, the torn site of the soft tissue 100 is strongly connected without any stress.

1-2-1. Method 1 for Treating Rotator Cuff Tear Using Net-Shaped Cylindrical Body

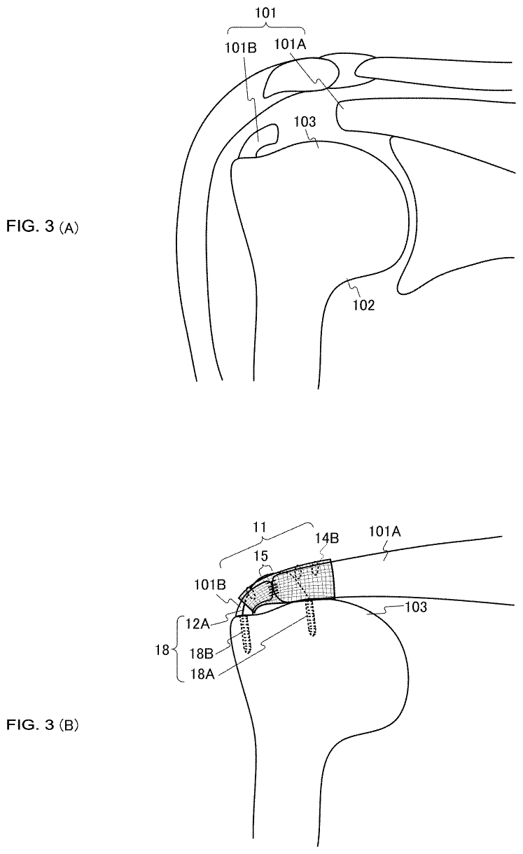

Referring to FIG. 3, a description will be given of a case where the rotator cuff tear is treated using the net-shaped cylindrical body 11. For example, as shown in FIG. 3(A), a case is assumed in which the rotator cuff 101 is torn into a rotator cuff 101A and a rotator cuff 101B near the site where the rotator cuff 101 is connected to the humerus 102. In such a case, a treatment practitioner performs the treatment of the rotator cuff tear by the following procedure. First, the rotator cuff 101A torn and separated from the humerus 102 is pulled toward the humerus 102. Then, the rotator cuff 101B is connected to the rotator cuff 101A according to the method described above in <1-2. Method for treating soft tissue using net-shaped cylindrical body>.

Subsequently, the rotator cuffs 101A and 101B are fixed to a bone head portion 103 of the humerus 102 using an engaging member 18. Note that the engaging member 18 includes anchors 18A and 18B provided at both ends of a thread member 12A. As shown in FIG. 3(B), the engaging member 18 is set such that the thread member 12A is passed through a mesh of the net-shaped cylindrical body 11 so as to be intertwined with the net-shaped cylindrical body 11. Note that various modes are conceivable as to how the thread member 12A is intertwined with the mesh of the net-shaped cylindrical body 11 by increasing or decreasing the number of times the thread member 12A is passed through the mesh of the net-shaped cylindrical body 11. Further, the engaging member 18 may be set such that the thread member 12A abuts on the net-shaped cylindrical body 11 along its outer circumferential surface without being intertwined with the net-shaped cylindrical body 11. In such a case, the thread member 12A and the net-shaped cylindrical body 11 are preferably stitched together with the thread member 14B.

From this state, the anchors 18A and 18B are driven into the bone head portion 103 of the humerus 102. In this manner, the net-shaped cylindrical body 11 is pressed against the bone head portion 103 of the humerus 102 and the rotator cuff 101 is fixed to the bone head portion 103 of the humerus 102.

As described above, when the net-shaped cylindrical body 11 is attached to the torn site of the rotator cuff 101, the load applied to the treating region 15 is dispersed to the surrounding area via the net-shaped cylindrical body 11. Further, the net-shaped cylindrical body 11 has stretchability. Thus, the net-shaped cylindrical body 11 allows the torn site of the rotator cuff 101 to deform without imposing an excessive load on the torn site of the rotator cuff 101. In this manner, the torn site of the rotator cuff 101 is strongly connected without any stress.

Note that, in the above-described case, the engaging member 18 does not penetrate the rotator cuff 101; however, the present invention also encompasses a case where the engaging member 18 penetrates the rotator cuff 101 for fixation.

1-2-2. Method 2 for Treating Rotator Cuff Tear Using Net-Shaped Cylindrical Body

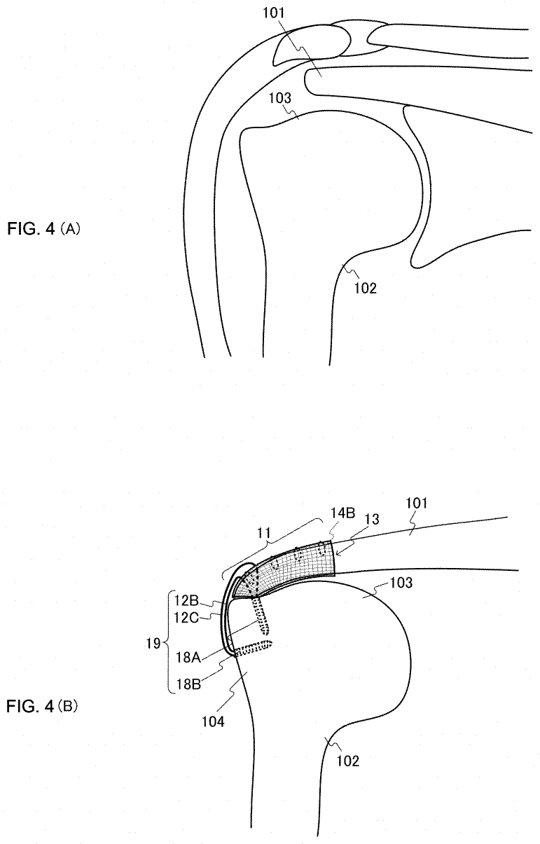

Referring to FIG. 4, a description will next be given of a case where the rotator cuff tear in another mode is treated using the net-shaped cylindrical body 11. For example, as shown in FIG. 4(A), a case is assumed in which the rotator cuff 101 was torn such that the substantially whole rotator cuff 101 is disconnected from the humerus 102. In such a case, the treatment practitioner performs the treatment of the rotator cuff tear by the following procedure. First, as show in FIG. 4(B), the rotator cuff 101 is inserted in the passage 13 of the net-shaped cylindrical body 11. Then, the rotator cuff 101 is engaged with the net-shaped cylindrical body 11 using the engaging member such as the thread member 14B. Then, the rotator cuff 101 is pulled toward the ceiling surface of the bone head portion 103 of the humerus 102.

Subsequently, the rotator cuff 101 is fixed to the bone head portion 103 of the humerus 102 using an engaging member 19. Note that the engaging member 19 includes the anchors 18A and 18B provided at both ends of two thread members 12B and 12C. As shown in FIG. 4(B), the engaging member 19 is set such that each of the thread members 12B and 12C is passed through the mesh of the net-shaped cylindrical body 11 and intertwined with the net-shaped cylindrical body 11. Note that various modes are conceivable as to how the thread members 12B and 12C are intertwined with the meshes of the net-shaped cylindrical body 11 by increasing or decreasing the number of times the thread members 12B and 12C are passed through the meshes of the net-shaped cylindrical body 11. Further, the engaging member 19 may be set such that the thread members 12B and 12C abut on the net-shaped cylindrical body 11 along its outer circumferential surface without being intertwined with the net-shaped cylindrical body 11. In such a case, the thread members 12B and 12C and the net-shaped cylindrical body 11 are preferably stitched together with the thread member 14B.

The anchor 18A is driven into the bone head portion 103 of the humerus 102. Further, the anchor 18B is driven near the greater tubercle 104 (or near the surgical neck) of the humerus 102. In this manner, the net-shaped cylindrical body 11 is pressed against the bone head portion 103 and the rotator cuff 101 is fixed to the bone head portion 103.

In this operation, the load applied to the torn site of the soft tissue is dispersed to the surrounding area via the net-shaped cylindrical body 11 by adjusting positions at which the thread members 12B and 12C are passed through the meshes of the net-shaped cylindrical body 11. Further, the net-shaped cylindrical body 11 has stretchability. Thus, the net-shaped cylindrical body 11 allows the torn site of the rotator cuff 101 to deform without imposing an excessive load on the torn site of the rotator cuff 101. In this manner, the torn site of the rotator cuff 101 is strongly connected without any stress.

Note that, in the above-described case, the engaging member 19 does not penetrate the rotator cuff 101; however, the present invention also encompasses a case where the engaging member 19 penetrates the rotator cuff 101 for fixation.

### 1-3. Material of Net-Shaped Body

Next, a specific material of the net-shaped body 10 including the knitted body will be described. The net-shaped body 10 is constituted of a titanium-tantalum (Ti—Ta) alloy including at least titanium (Ti) and tantalum (Ta).

The alloy constituting the net-shaped body 10 is not particularly limited as long as it contains at least titanium and tantalum. The alloy may also include an element other than titanium and tantalum. For example, the alloy constituting the net-shaped body 10 may contain tin (Sn) in addition to titanium and tantalum and, in such a case, more excellent mechanical properties can be obtained.

Specifically, the titanium-tantalum alloy containing tin (Sn) in an embodiment of the present invention preferably contains 15 atomic percent (at %) or more to 27 at % or less of tantalum and 0 at % or more to 8 at % or less of tin, where the total is 100 at %, with remainder being titanium and unavoidable impurities. Such an alloy can achieve not only more excellent mechanical properties, that is, a high tensile strength, a low Young's modulus, and an appropriate elastic limit, but also high biocompatibility.

Note that a lower limit value of the content of tin (Sn) may be 0 at % as described above. This is because the titanium-tantalum alloy having the mechanical properties (the Young's modulus, the tensile strength, and the elastic deformation strain) required for the net-shaped body 10 can be obtained without adding Sn if the content of Ta is 15 at % or more.

However, tin (Sn) is preferably added to the titanium-tantalum alloy to further improve the mechanical properties. For example, from the standpoint of the superelastic effect of the titanium-tantalum alloy, tin (Sn) has a function of inhibiting precipitation of an ω□phase that may cause an increase in the Young's modulus and thus of improving the superelastic effect of the titanium-tantalum alloy. The superelastic effect enables a flexible response to unintended deformation. Thus, tin (Sn) is preferably added to the titanium-tantalum alloy. Note that the content of Sn is preferably 1 at % or more where the total titanium alloy is 100 at % to sufficiently exhibit the w phase inhibition function described above.

Further, the aforementioned titanium-tantalum alloy containing tin (Sn) has very little amount of elution of metal ions of constituent elements, Ti, Ta, and Sn. Further, it has excellent corrosion resistance, low cytotoxicity, and high biocompatibility. The titanium-tantalum alloy containing tin (Sn) is a non-magnetic material hardly magnetized by an external magnetic field and is thus very unlikely to adversely affect a medical equipment (MRI, etc.) adversely affected by magnetism. The titanium-tantalum alloy containing tin (Sn) has a high elasticity, an appropriate rigidity, and high processability. That is, the aforementioned titanium-tantalum alloy containing tin (Sn) has lower cytotoxicity and more excellent magnetic properties, corrosion resistance, mechanical properties, and processability than a conventional titanium alloy.

1-3-1. Imaging Properties of Material of Net-Shaped Body

Further, for example, an implant used in the body is conventionally constituted of a stainless steel such as SUS316L, a nickel-titanium (Ni—Ti) alloy as a superelastic alloy, or the like to obtain required mechanical properties (the tensile strength, the Young's modulus, and the elastic limit). However, these materials have a low X-ray absorbance and thus is hardly photographed in an X-ray image. On the other hand, the titanium-tantalum alloy, which has the tensile strength and the Young's modulus equivalent to those of the nickel-titanium alloy as a superelastic alloy, has a property of having a higher X-ray absorbance (X-ray impermeability) by containing tantalum with larger atomic weight. Thus, the net-shaped body 10 constituted of the titanium-tantalum alloy has excellent imaging properties at the time of photographing an X-ray image.

1-3-2. Plastic Deformation of Material of Net-Shaped Body

The titanium-tantalum alloy has the appropriately lower elastic limit than the nickel-titanium alloy. Thus, the net-shaped body 10 constituted of the titanium-tantalum alloy can be plastically deformed according to the shape of an attachment site, while providing the strength and flexibility equivalent to those of the nickel-titanium alloy.

1-3-3. Surface Film of Material of Net-Shaped Body

Further, when a polymer coated layer made of a polymer having biocompatibility is disposed on the surface of the titanium-tantalum alloy, the polymer coated layer functioning as a protective film can improve the corrosion resistance of the titanium-tantalum alloy. Thus, the net-shaped body 10 constituted by the linear member made of the titanium-tantalum alloy covered with the polymer coated layer has the improved corrosion resistance. Thus, it is safe to indwell the net-shaped body 10 in the body for a long period of time. Further, the net-shaped body 10 constituted by the linear member made of the titanium-tantalum alloy covered with the polymer coated layer can easily slide on the living tissue. That is, the polymer coated layer imparts lubricity to the net-shaped body 10. In particular, when the polymer coated layer is formed of a hydrophilic polymer, the net-shaped body 10 can further easily slide on the living tissue. This allows the net-shaped body 10 to easily move in the body without imposing a load on the living tissue, making it easy to dispose the net-shaped body 10 at an affected site.

Further, when a hydroxyapatite coated layer made of hydroxyapatite is disposed on the surface of the titanium-tantalum alloy, the hydroxyapatite coated layer having sufficient biocompatibility can further improve the biocompatibility of the titanium-tantalum alloy. Thus, the net-shaped body 10 constituted by the linear member made of the titanium-tantalum alloy covered with the hydroxyapatite coated layer has the improved biocompatibility. Thus, it is safe to indwell the net-shaped body 10 in the body for a long period of time.

Further, when an oxide film layer is disposed on the surface of the titanium-tantalum alloy, the oxide film layer functioning as a protective film can improve the corrosion resistance of the titanium-tantalum alloy. Thus, the net-shaped body 10 constituted by the linear member made of the titanium-tantalum alloy covered with the oxide film layer has the improved corrosion resistance. Thus, it is safe to indwell the net-shaped body 10 in the body for a long period of time.

Note that the thickness of the polymer coated layer, hydroxyapatite coated layer, or oxide film layer covering the surface of the titanium-tantalum alloy in the present invention is controlled in the production stage.

1-3-4. Other Materials Constituting Net-Shaped Body

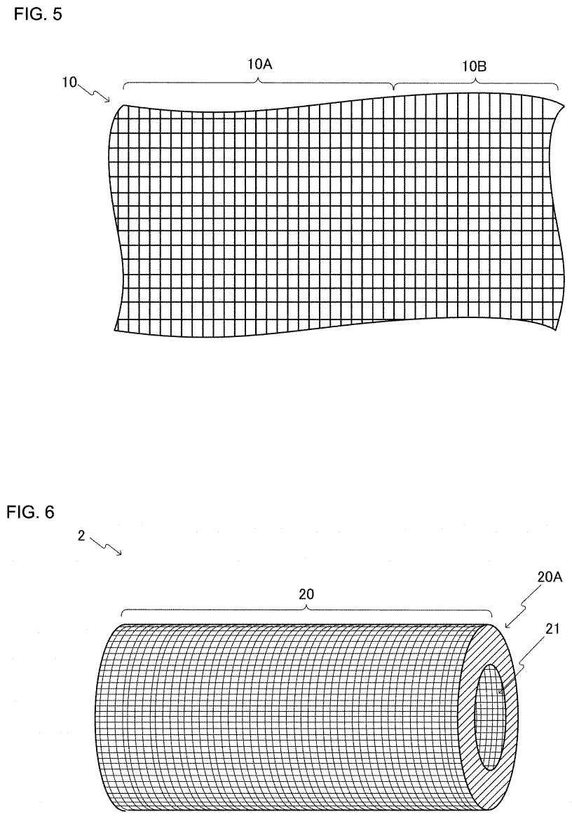

Further, the net-shaped body 10 may be formed of the titanium-tantalum alloy and the polymer. Examples of the net-shaped body 10 containing the polymer (hereinafter referred to as “polymer containing net-shaped body”) may include a product in which a partial region 10A is constituted of the titanium-tantalum alloy and a remaining region 10B is constituted of the polymer as shown in FIG. 5, a knitted body in which the linear member made of the titanium-tantalum alloy and the linear member made of the polymer are alternately knitted next to each other, a knitted body in which a warp thread is constituted by the linear member made of the titanium-tantalum alloy and a weft thread is constituted by the linear member made of the polymer, and a product in which the sheet-shaped net-shaped body 10 made of the titanium-tantalum alloy and a sheet made of the polymer are formed in a layer shape.

Note that, as the polymer described above, for example, a polymer for a super fabric, such as para-aramid, ultrahigh molecular weight polyethylene, polyarylate (a polyester liquid crystal polymer), and polyetheretherketone, is preferable. However, the polymer is not limited thereto and may be other polymers. Examples of other polymers may include polyester, acryl, nylon, vinylon, polypropylene, polyvinyl chloride, polyethylene, polyvinylidene, polyurethane, polyamide, polyvinyl alcohol, polyethylene oxide, polyacrylamide, polyether, polycarboxylic acid, cellulose polymer, polychlal, rayon, polynosic, cupra, acetate, triacetate, promix, and lyocell.

The polymer containing net-shaped body preferably has lubricity. This is because, when lubricity is imparted to the polymer containing net-shaped body, the polymer containing net-shaped body brought into contact with the soft tissue in the living body can slide on the soft tissue without imposing a load on the soft tissue. Note that the term “lubricity” used herein refers to a property in which the polymer containing net-shaped body brought into contact with the living body can slide on the living body without being integrated therewith. For imparting lubricity to the polymer containing net-shaped body, the polymer portion of the polymer containing net-shaped body is preferably formed of a hydrophilic polymer.

Further, the net-shaped body 10 may be formed of the titanium-tantalum alloy and a biodegradable material having biocompatibility. Examples of the biodegradable material may include a biodegradable plastic, biodegradable metal, and the like. The biodegradable plastic is hydrolyzed by an acid or alkali in the living body and excreted from the body, and examples thereof may include a material constituted of polyglycolic acid, polydioxanone, or polylactic acid. Further, examples of the biodegradable metal may include magnesium (Mg), and a magnesium alloy.

Examples of the net-shaped body 10 containing the biodegradable material (hereinafter referred to as “biodegradable material containing net-shaped body”) may include, similar to above, a product in which the partial region 10A is constituted of the titanium-tantalum alloy and the remaining region 10B is constituted of the polymer, a product in which the linear member made of the titanium-tantalum alloy and the linear member made of the biodegradable material are alternately knitted next to each other, a knitted body in which a warp thread is constituted by the linear member made of the titanium-tantalum alloy and a weft thread is constituted by the linear member made of the biodegradable material, and a product in which the sheet-shaped net-shaped body 10 made of the titanium-tantalum alloy and the sheet made of the biodegradable material are formed in a layer shape.

In an initial stage of attaching the biodegradable material containing net-shaped body to the soft tissue failure site, the biodegradable material containing net-shaped body is in a state of high rigidity as the hydrolysis of the biodegradable material is not sufficiently proceeded. There still remain many portions of the biodegradable materials with high rigidity in the biodegradable material containing net-shaped body. However, the hydrolysis of the biodegradable material portions gradually proceeds with the lapse of time and the biodegradable material portions are gradually removed from the body. Thus, the biodegradable material containing net-shaped body gradually becomes more easily deformable. As describe above, the biodegradable material containing net-shaped body is useful in the case where it is desirable that the net-shaped body 10 having high rigidity at the initial stage is made easily deformable with the lapse of time.

### 2. Second Embodiment

Referring FIG. 6, a medical member 2 in a second embodiment of the present invention will be described below. The medical member 2 is constituted by a net-shaped cylindrical body 20 formed in a cylindrical shape. The net-shaped cylindrical body 20 is, for example, as shown in FIG. 6, constituted by a main body 20A formed by a net-shaped member in a cylindrical shape and a passage 21 penetrating the main body. The soft tissue failure site is inserted in the passage 21.

While the medical member 1 is formed by the net-shaped body 10 in the sheet shape, the medical member 2 is not formed by the net-shaped body 10 in the sheet shape. The medical member 2 has a configuration in which a through-hole is disposed in the main body 20A formed by the net-shaped member in a three-dimensional shape.

### 3. Third Embodiment

### 3-1. Overall Configuration

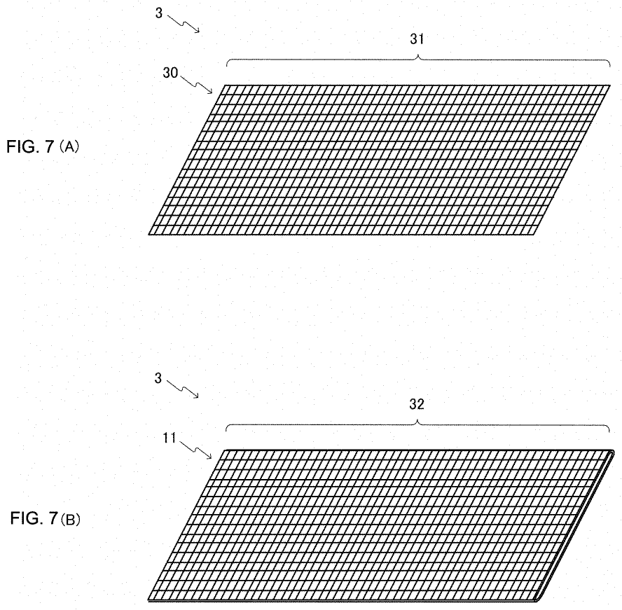

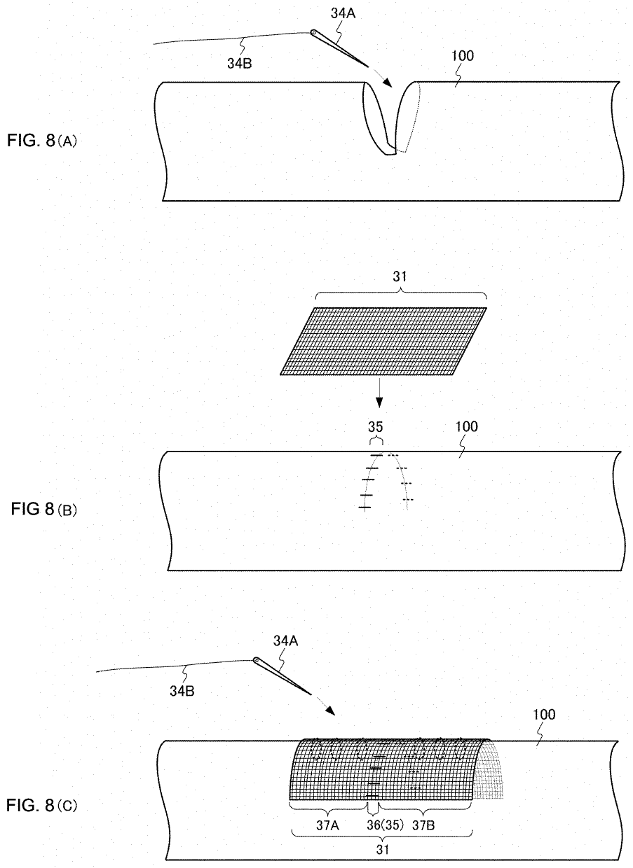

Referring to FIG. 7 and FIG. 8, a medical member 3 in a third embodiment of the present invention will be described below. As shown in FIG. 7(A), the medical member 3 is constituted by a net-shaped sheet body 31 formed by a net-shaped body 30. The net-shaped body 30, including its material, is similar to the net-shaped body 10 described above, and since it has already been described above, thus a further explanation thereof is omitted herein.

Note that, as shown in FIG. 7(B), the net-shaped cylindrical body 11 in the first embodiment of the present invention may be plastically deformed by folding and pressing to form a double-layered net-shaped sheet body 32. Such a product is also included in the technical idea of the net-shaped sheet body.

### 3-2. Method for Using Net-Shaped Sheet Body

A method for using the net-shaped sheet body 31 will be described with reference to FIG. 8. The net-shaped sheet body 31 is, for example, attached to the soft tissue failure site. A description will now be given of a case where the net-shaped sheet body 31 is attached to the partially torn soft tissue 100 (see FIG. 8(A)). First, as shown in FIG. 8(A), the torn site of the soft tissue 100 is stitched using a needle member 34A and a thread member 34B by the treatment practitioner.

Next, as shown in FIG. 8(B), the net-shaped sheet body 31 is put on the soft tissue 100 so as to cover a treating region 35. Note that the treating region 35 refers to a region occupied by a treating member (for example, the thread member 34B) at the torn site and a surrounding area thereof of the soft tissue when the torn site of the soft tissue is treated by stitching or the like. In this operation, as shown in FIG. 8(C), the net-shaped sheet body 31 is disposed across the treating region 35. Thus, it can be said that the net-shaped sheet body 31 includes a first region 36 that covers the treating region 35 and second regions 37A and 37B that cover the surrounding regions of the treating region 35.

The engagement of the second regions 37A and 37B with the soft tissue 100 can be achieved, for example, as shown in FIG. 8(C), by stitching the net-shaped sheet body 31 and the soft tissue 100 together using the needle member 34A and the thread member 34B, without being limited thereto. The engagement of the net-shaped sheet body 31 with the soft tissue 100 may be achieved, for example, by other engaging members such as the skin stapler.

As described above, when the net-shaped sheet body 31 is attached to the torn site of the soft tissue 100, a load applied to the treating region 35 is dispersed to the surrounding area via the net-shaped sheet body 31. Further, the net-shaped sheet body 31 has stretchability. Thus, the net-shaped sheet body 31 allows the torn site of the soft tissue 100 to deform without imposing an excessive load on the torn site of the soft tissue 100. In this manner, the torn site of the soft tissue 100 is strongly connected without any stress.

### 4. Fourth Embodiment

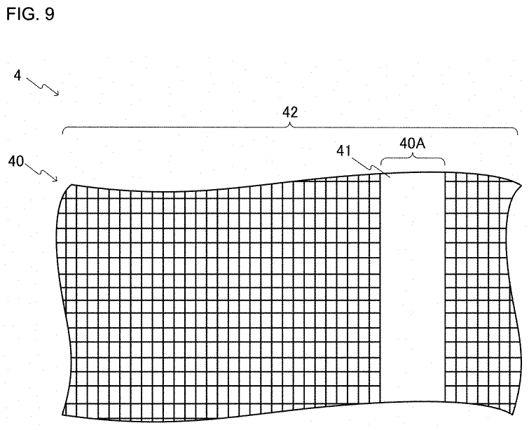

Referring to FIG. 9, a medical member 4 in a fourth embodiment of the present invention will be described below. As shown in FIG. 9, the medical member 4 includes a mixed body 42 in which a sheet body 41 is included in a portion of a net-shaped body 40. The sheet body 41 is, for example, as shown in FIG. 9, disposed so as to constitute a partial region 40A in the net-shaped body 40.

The mixed body 42 can be used to form a product similar to the net-shaped cylindrical body 11 or the net-shaped sheet body 31. The net-shaped cylindrical body or the net-shaped sheet body formed by the mixed body 42 is also included in the scope of the present invention. Further, the net-shaped cylindrical body or the net-shaped sheet body formed by the mixed body 42 can be used according to the use methods described in the first embodiment and the second embodiment.

Further, materials of the net-shaped body 40 and the sheet body 41 in the mixed body 42 are similar to those of the net-shaped body 10 described in <1-3. Material of net-shaped body> and the description in <1-3. Material of net-shaped body> can be applied to the net-shaped body 40 and the sheet body 41 in the mixed body 42.

Further, the sheet body 41 in the mixed body 42 can be formed of the polymer or the biodegradable material having biocompatibility. Note that, as an example of the polymer described above, a polymer for a super fabric, such as para-aramid, ultrahigh molecular weight polyethylene, polyarylate (a polyester liquid crystal polymer), and polyetheretherketone, is preferable. However, the polymer is not limited thereto and may be other polymers. Examples of other polymers may include polyester, acryl, nylon, vinylon, polypropylene, polyvinyl chloride, polyethylene, polyvinylidene, polyurethane, polyamide, polyvinyl alcohol, polyethylene oxide, polyacrylamide, polyether, polycarboxylic acid, cellulose polymer, polychlal, rayon, polynosic, cupra, acetate, triacetate, promix, and lyocell.

Further, for example, lubricity may be imparted to any of the net-shaped body 40, the sheet body 41, and the mixed body 42. This is because any of the net-shaped body 40, the sheet body 41, and the mixed body 42 with lubricity can be brought into contact with the soft tissue in the living body without imposing a load on the soft tissue. Note that the term “lubricity” used herein refers to a property in which any of the net-shaped body 40, the sheet body 41, and the mixed body 42 brought in contact with the living body can slide on the living body without being integrated therewith. Any of the net-shaped body 40, the sheet body 41, and the mixed body 42 with lubricity is preferably formed of a hydrophilic polymer.

### 5. Fifth Embodiment

### 5-1. Overall Configuration

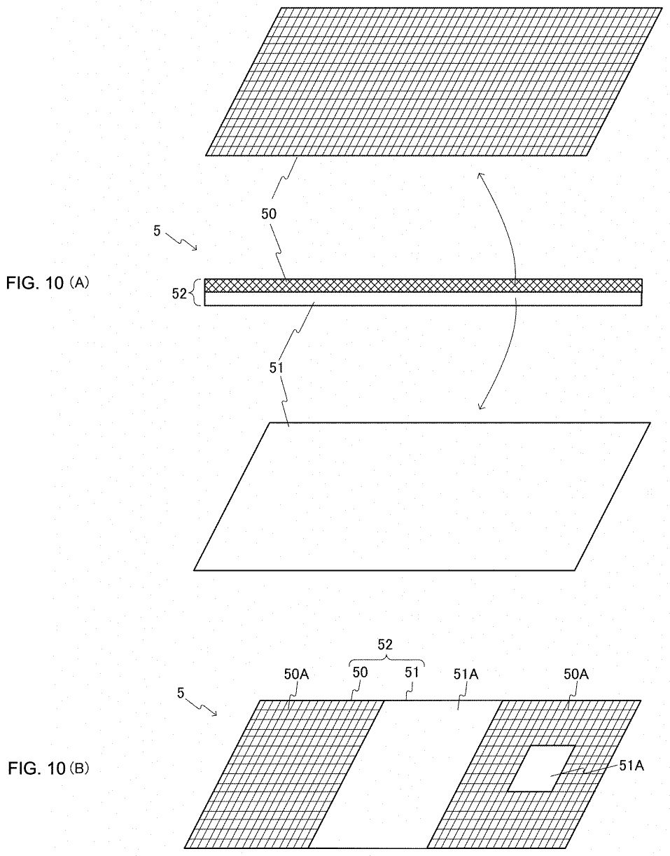

Referring to FIG. 10, a medical member 5 in a fifth embodiment of the present invention will be described below. As shown in FIG. 10(A), the medical member 5 is constituted by a layer body 52 in which a net-shaped body 50 and a sheet body 51 are formed in a layer shape. The net-shaped body 50, including its material, is similar to the net-shaped bodies 10, 30, and 40 described above, and since it has already been described above, thus a further explanation thereof is omitted herein.

Further, the sheet body 51 is formed of a non-integrated biomaterial having a property of not being integrated with a biotissue. Further, the sheet body 51 may have lubricity. Examples of the non-integrated biomaterial may include a polymer which contains at least one component of fluoroethylene, fluoropropylene, and fluoro-polyethylene glycol and is derived from any component of lactide, glycolide, caprolactone, valerolactone, carbonate, dioxepanone, ethylene glycol, ethylene oxide, ester amide, γ-hydroxyvalerate, β-hydroxypropionate, α-hydroxy acid, hydroxybutyrate, poly(orthoester), hydroxyalkanoate, tyrosine carbonate, poly(imidocarbonate), poly(iminocarbonate), polyurethane, a polyacid anhydride, a polymer drug, polyolefin, polyester, nylon, polyamide, polybutester, and polyaryletherketone, and a combination thereof. Further, the non-integrated biomaterial may be a non-crosslinkable silicone polymer, which is a lubricating silicone sheet composition or a lubricating silicone coating composition selected from polydimethylsiloxane, polyalkylmethylsiloxane, polydiethylsiloxane, polyfluoropropylmethylsiloxane, polyocthylmethylsiloxane, polytetradecylmethylsiloxane, polyoctadecylmethylsiloxane, and polyalkylmethyldimethylsiloxane (for example, polyhexadecymethylsiloxane-dimethylsiloxane). Further, the non-integrated biomaterial may be a polymer for a super fabric, such as para-aramid in which a 2-methacryloyloxyethyl phosphorylcholine (MPC) polymer is subjected to graft coating, ultrahigh molecular weight polyethylene, polyarylate (a polyester liquid crystal polymer), and polyetheretherketone.

The layer body 52 can be used to form a product similar to the net-shaped cylindrical body 11 or the net-shaped sheet body 31. The net-shaped cylindrical body or the net-shaped sheet body formed by the layer body 52 is also included in the scope of the present invention. Further, the net-shaped cylindrical body or the net-shaped sheet body formed by the layer body 52 can be used according to the use methods described in the first embodiment and the second embodiment.

5-2-1. Method 1 for Treating Inguinal Hernia Using Layer Body

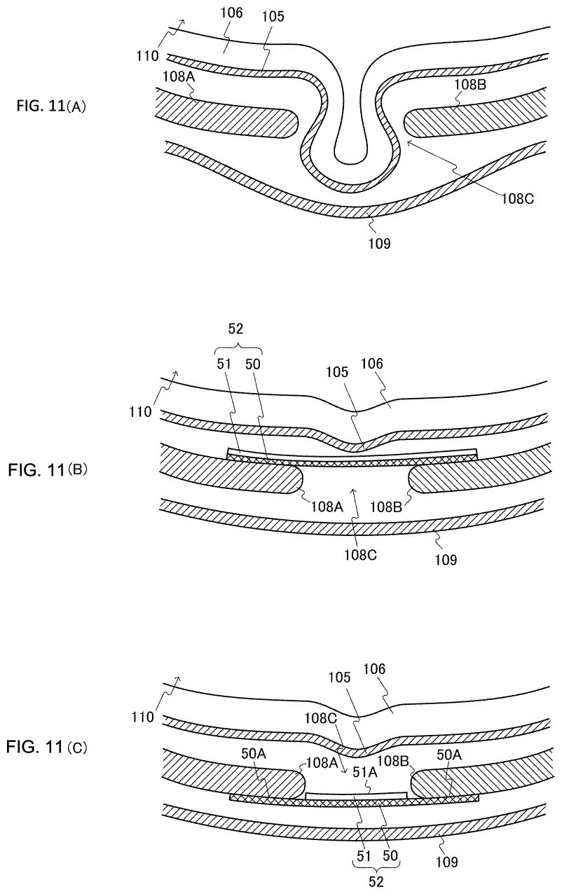

Referring to FIG. 11, an example of a method of using the layer body 52 for treating inguinal hernia will be described. For example, as shown in FIG. 11(A), a case is assumed in which parts of the peritoneum 105 and the intestine 106 protrude through an opening 108C between the fasciae 108A and 108B in the groin toward a side of the skin 109. This is referred to as an inguinal hernia.

In order to treat the inguinal hernia, the opening 108C needs to be closed by the layer body 52. In this operation, the layer body 52 is disposed on a side facing an internal body 110 in a thickness direction of the fasciae 108A and 108B such that the net-shaped body 50 of the layer body 52 abuts on portions of the fasciae 108A and 108B on the internal body 110 side. As a result, the peritoneum 105 and the intestine 106 are prevented from protruding through the opening 108C toward the skin 109 side. In this manner, the treatment of the inguinal hernia is completed.

Further, although the sheet body 51 is disposed on the side where the sheet body 51 can come into contact with the peritoneum 105, the sheet body 51 formed of the non-integrated biomaterial is not integrated with the peritoneum 105 while being in contact with it. Thus, the layer body 52 disposed on the opening 108C as described above is not integrated with a tissue, such as the peritoneum 105, with which the layer body 52 is not desired to be integrated.

5-2-2. Method 2 for Treating Inguinal Hernia Using Layer Body

Further, the layer body 52 may be disposed on the skin 109 side in the thickness direction of the fasciae 108A and 108B. In such a case, as shown in FIG. 10(B), the layer body 52 may be configured such that the sheet body 51 is disposed on a part of a plane region of the net-shaped body 50 to form a layer. In a case where such a layer body 52 is attached to the fasciae 108A and 108B, as shown in FIG. 11(C), the sheet body 51 of the layer body 52 is disposed near the opening 108C so as to face the peritoneum 105 through the opening 108C. Further, in FIG. 11(C), the sheet body 51 of the layer body 52 is formed in a size that prevents the sheet body 51 from abutting on the fasciae 108A and 108 and then disposed. However, the configuration of the sheet body 51 is not limited thereto and the sheet body 51 may be formed in a size that causes a part of the sheet body 51 to abut on the fasciae 108A and 108. Further, the net-shaped body 50 of the layer body 52 is disposed so as to abut on the parts of the fasciae 108A and 108B on the skin 109 side.

That is, the layer body 52 has a peritoneum facing surface 51A and a fascia abutting surface 50A. The peritoneum facing surface 51A is a surface of the sheet body 51 that faces the peritoneum 105. The fascia abutting surface 50A is a surface of the net-shaped body 50 on the side where the sheet body 51 of the net-shaped body 50 is laminated, which abuts on the parts of the fasciae 108A and 108B on the skin 109 side.

As shown in FIG. 10(B), the peritoneum facing surface 51A may be disposed between two fascia abutting surfaces 50A (see the middle part of FIG. 10(B)) or disposed as to be surrounded by the fascia abutting surface 50A (see the right part of FIG. 10(B)). The aforementioned layer body 52 attached in the above manner is not integrated with a tissue, such as the peritoneum 105, with which the layer body 52 is not desired to be integrated.

Note that the layer body 52 can be applied to any soft tissue failure site. The layer body 52 is preferably applied to a part where the layer body 52 is not desired to be integrated with a surrounding tissue of the soft tissue failure site.

### 5-3. Modification of Layer Body

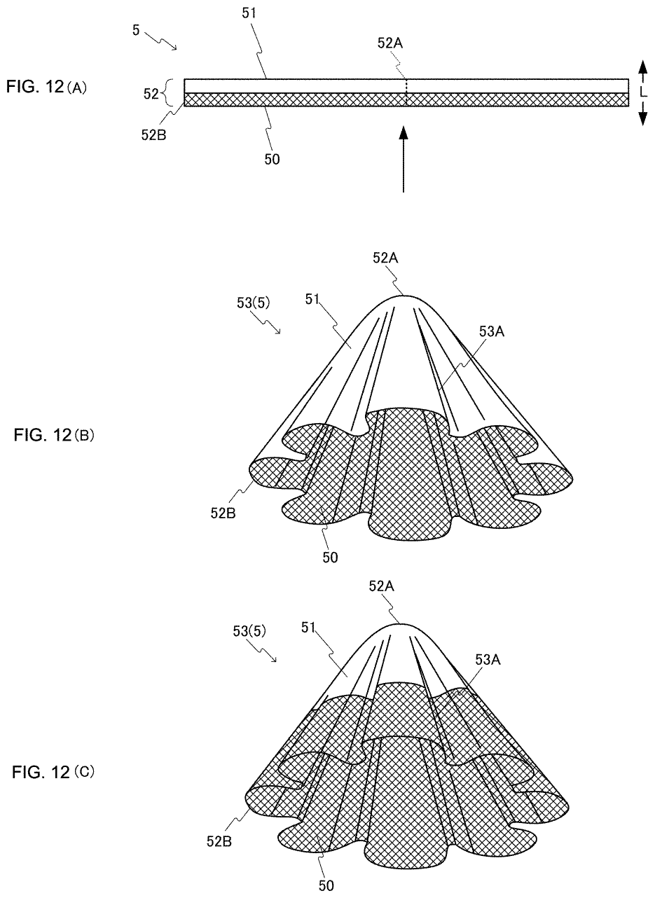

Referring to FIG. 12, a modification of the medical member 5 in the fifth embodiment of the present invention will be described below. As shown in FIG. 12(A), a substantial center portion 52A of the layer body 52 is deformed so as to project to the sheet body 51 side in a lamination direction of the net-shaped body 50 and the sheet body 51 (see an arrow L direction in FIG. 12(A)) as compared with an outer edge portion 52B of the layer body 52. In this manner, a projecting body 53 as a modification of the medical member 5 is formed. The projecting body 53 includes the sheet body 51 in its outside and the net-shaped body 50 in its inside.

As shown in FIG. 12(B), the projecting body 53 has, for example, an approximately conical shape. Further, the projecting body 53 includes a plurality of pleats 53A extending in a height direction thereof on a peripheral surface of the conical shape. The plurality of pleats 53A are arranged in a circumferential direction of the projecting body 53. The net-shaped body 50 constituted of the titanium-tantalum alloy having an appropriately low elastic limit makes it easy to produce the projecting body 53 described above from the layer body 52. Note that the projecting body 53 may be formed in a shape other than the conical shape as long as at least a part of the layer body 52 inside the outer edge portion 52B projects to the sheet body 51 side in the lamination direction of the net-shaped body 50 and the sheet body 51 (see an arrow L direction in FIG. 12(A)) as compared with the outer edge portion 52B of the layer body 52. Further, as shown in FIG. 12(C), the projecting body 53 may be configured such that the sheet body 51 is disposed on a part of a surface side region of the net-shaped body 50 to form a layer.

### 5-4. Method for Treating Inguinal Hernia Using Projecting Body

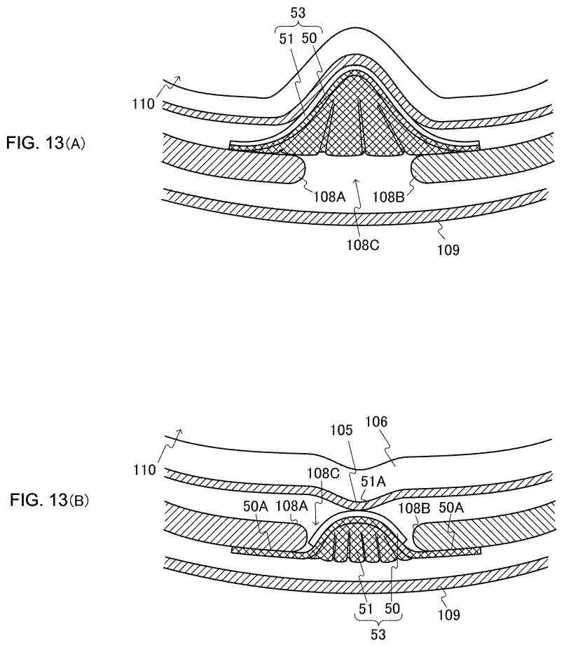

Referring to FIG. 13, an example of a method of using the projecting body 53 for treating the inguinal hernia will be described. For treating the inguinal hernia shown in FIG. 11(A), the opening 108C needs to be closed by the projecting body 53. In this operation, as shown in FIG. 13(A), the projecting body 53 is attached such that the net-shaped body 50 of the projecting body 53 abuts on the fasciae 108A and 108B on the internal body 110 side in the thickness direction of the fasciae 108A and 108B and the sheet body 51 of the projecting body 53 faces the peritoneum 105. As a result, the projecting body 53 projects toward the internal body 110 side in the thickness direction of the fasciae 108A and 108B. Then, the peritoneum 105 and the intestine 106 are pressed back toward the internal body 110 side by the projecting body 53 and prevented from coming out to the skin 109 side through the opening 108C. In this manner, the treatment of the inguinal hernia is completed.

The projecting body 53 may be disposed on the skin 109 side in the thickness direction of the fasciae 108A and 108B. In such a case, the projecting body 53 in which the sheet body 51 is disposed on the part of the surface side region of the net-shaped body 50 to form a layer, shown in FIG. 12(C), is used. As shown in FIG. 13(B), the sheet body 51 of the projecting body 53 is disposed near the opening 108C so as to face the peritoneum 105. Then, the sheet body 51 of the projecting body 53 is formed in a size that prevents the sheet body 51 from abutting on the fasciae 108A and 108 and then disposed. However, the configuration of the sheet body 51 is not limited thereto and the sheet body 51 may be formed in a size that causes a part of the sheet body 51 to abut on the fasciae 108A and 108. Further, the net-shaped body 50 of the projecting body 53 is disposed so as to abut on the parts of the fasciae 108A and 108B on the skin 109 side.

The projecting body 53 described above has the peritoneum facing surface 51A and the fascia abutting surface 50A. The peritoneum facing surface 51A and the fascia abutting surface 50A of the projecting body 53 can be described in a similar manner to that for the fascia abutting surface 50A and the peritoneum facing surface 51A of the layer body 52 described above. Since it has already been described above, thus, a further explanation thereof is omitted herein.

### 5-5. Other Modifications

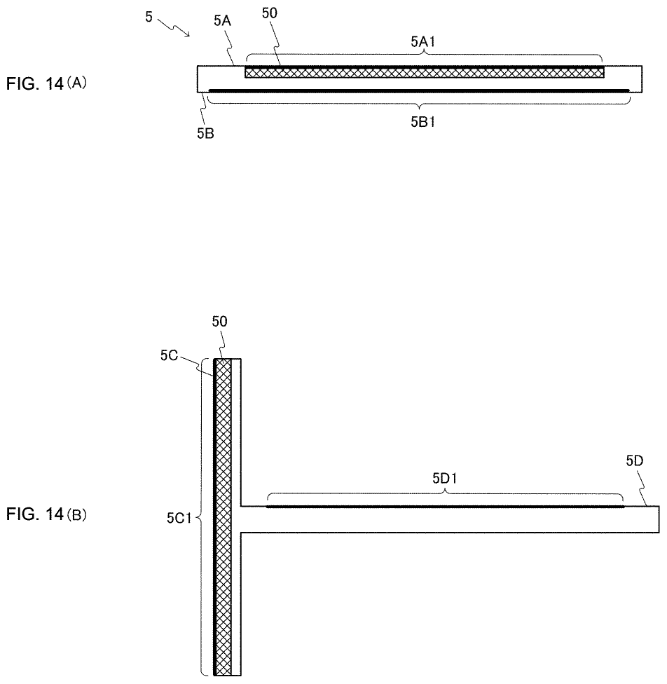

Note that the medical member 5 is not limited to the layer body 52 and may have another configuration which includes a portion to be integrated with a biotissue and a portion not to be integrated with a biotissue. That is, the medical member 5 may be constituted by the net-shaped body 50 and other constituent elements as long as, as a result, a surface region 5A1 constituting at least a part of a surface 5A on one side of the medical member 5 is constituted of a material capable of being integrated with a biotissue and a surface region 5B1 constituting at least a part of a surface 5B on the other side (on the side opposite to one side of the surface 5A) of the medical member 5 is constituted of a material not to be integrated with a biotissue, as shown in FIG. 14(A). In such a case, any material may be interposed between the surface region 5A and the surface region 5B. Specifically, the surface region 5A is constituted, for example, by the surface of the net-shaped body 50. Further, the surface region 5B may be constituted, for example, by coating the corresponding surface region with the non-integrated biomaterial or by a surface of a constituent element formed of the non-integrated biomaterial.

Further, the medical member 5 is not limited to the configuration in which the surface constituted of the material capable of being integrated with a biotissue (hereinafter referred to as “biotissue integrated surface”) is disposed on the side opposite to the surface constituted of the material not to be integrated with a biotissue (hereinafter referred to as “biotissue non-integrated surface”), and the former surface may be disposed on a surface other than the opposite surface. For example, as shown in FIG. 14(B), in the medical member 5 having a T-shaped cross section, a biotissue integrated surface region 5C1 may be disposed on a tip surface 5C in the height direction of the T-shape and a biotissue non-integrated surface region 5D1 may be disposed on a circumferential surface 5D of a T-shape portion extending in the height direction of the T-shape.

Note that, in the first to fifth embodiments of the present invention, a portion formed of the polymer is referred to as a polymer portion. The polymer portion refers to any portion formed of the polymer in the medical member of the present invention, without being limited to the polymer coated layer coating the linear member or the net-shaped body or the sheet body formed of the polymer.

Further, the description above has been given of the method for treating the tear of the soft tissue such as the rotator cuff and the hernia using the medical member of the present invention. However, an application range of the present invention is not limited thereto. In addition, a tear in the nerve can be treated using the medical member of the present invention according to the examples of the treatment methods described above.

### 6. Sixth and Seventh Embodiments

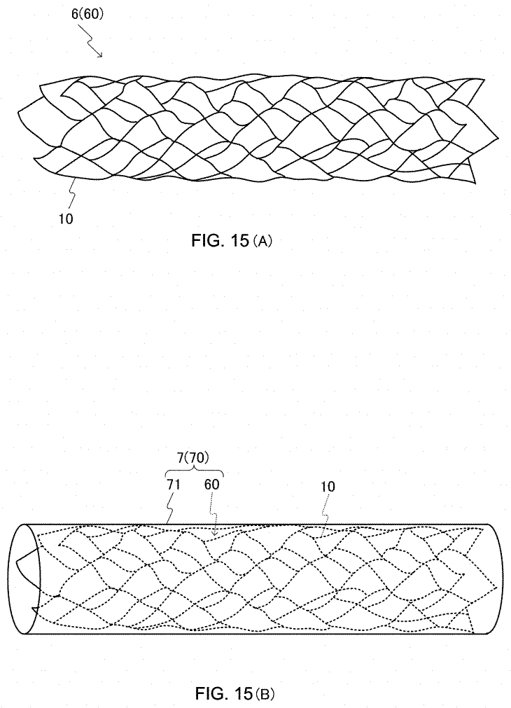

Referring to FIG. 15, medical members 6 and 7 in sixth and seventh embodiments of the present invention will be described below. As shown in FIG. 15(A), the medical member 6 is a stent 60 in which the net-shaped body 10 is formed in a cylindrical shape. The stent 60 expands a tubular portion in the soft tissue from the inside of the tube. Examples of the tubular portion in the soft tissue may include a blood vessel, trachea, esophagus, duodenum, large intestine, and biliary tract.

The stent 60 has biocompatibility and stretchability. The stretchability of the stent 60 can be controlled by a density of the mesh, a direction of the mesh, and the like of the knitted body constituting the net-shaped body 10.

Further, as shown in FIG. 15(B), the medical member 7 is a stent graft 70 that includes the stent 60 in its inside. The stent graft 70 is constituted by the stent 60 and a cylindrical artificial blood vessel 71. The stent 60 is attached to the inside (an inner circumferential surface) of the artificial blood vessel 71.

### 7. Eighth Embodiment

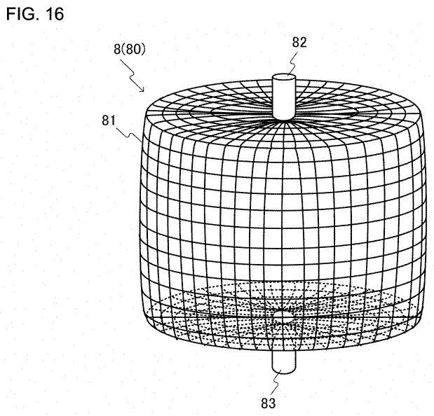

Referring to FIG. 16, a medical member 8 in an eight embodiment of the present invention will be described below. As shown in FIG. 16, the medical member 8 is an embolization member 80 formed by a net-shaped body 81 in a substantially cylindrical shape. The net-shaped body 81 is a net-shaped material. The inside of the embolization member 80 is also filled with the net-shaped body 81. The embolization member 80 blocks a blood flow in a blood vessel. The embolization member 80 is provided, for example, in the aneurysm or the like. The embolization member 80 disposed in the aneurysm or the like closes the aneurysm or the like. In this manner, the embolization member 80 blocks a blood flow into the aneurysm or the like.

The embolization member 80 includes an end screw 82 projected from a center of one bottom surface of the cylinder and a marker band 83 projected from a center of the other bottom surface of the cylinder. The end screw 82 is engaged with a tip of a pusher wire (not illustrated). The pusher wire is an instrument for pushing out the embolization member 80 into the aneurysm or the like from a tip of a catheter for angiography (not illustrated). The marker band 83 is disposed to confirm that the embolization member 80 is located on a target site in the catheter for angiography. The embolization member 80 is moved by the pusher wire while the position of the embolization member 80 is confirmed by the marker band 83.

Note that the shape of the embolization member is not limited to the cylindrical shape and the embolization member may be formed in other shapes as long as it is formed such that the blood flow into the aneurysm or the like can be blocked by the net-shaped body 81.

### 8. Ninth and Tenth Embodiments

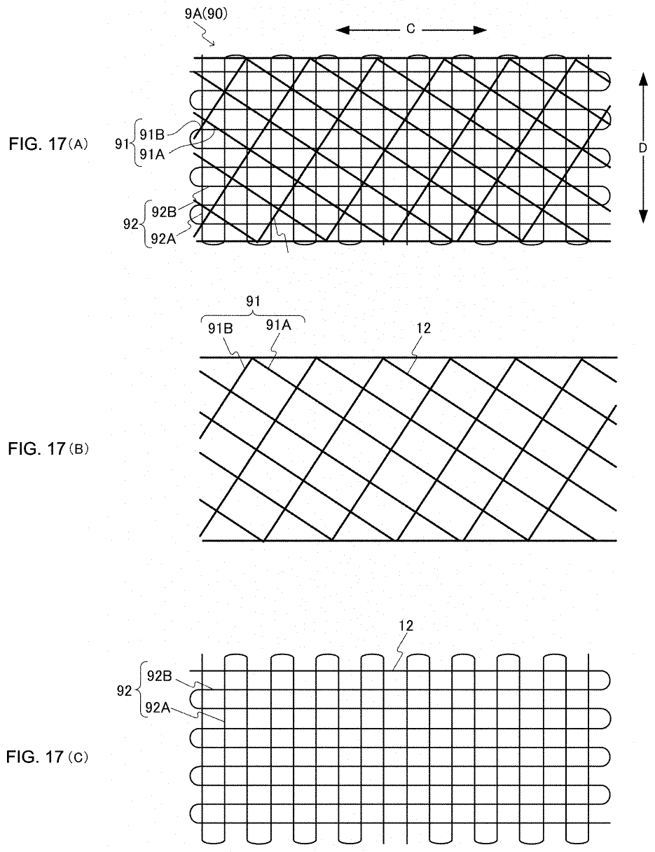

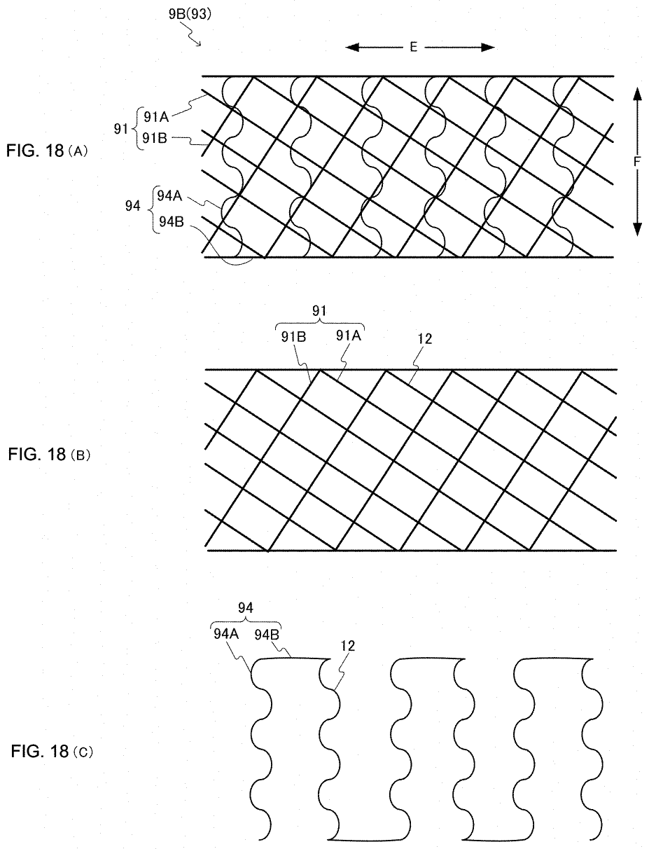

Referring to FIGS. 17 and 18, medical members 9A and 9B in ninth and tenth embodiments of the present invention will be described below. First, referring to FIG. 17, the medical member 9A in the ninth embodiment of the present invention will be described. The medical member 9A is constituted by a knitted body 90. As shown in FIG. 17(A), the knitted body 90 is constituted by two knitted body elements 91 and 92. The knitted body element is knitted by a predetermined knitting mode using, for example, the linear member 12 or the like. The linear member 12 has already been described above, and thus a further explanation thereof is omitted herein. Examples of the predetermined knitting mode may include plain weaving, twilled weaving, plain dutch weaving, twilled dutch weaving, and stockinette stitch.

As shown in FIGS. 17(A) and (B), the knitted body element 91 is formed such that a warp thread 91A and a weft thread 91B are knitted so as to intersect each other with predetermined inclined angles relative to a length direction C and a width direction D of the knitted body 90. Thus, the knitted body element 91 is stretchable in the length direction C and the width direction D of the knitted body 90.

As shown in FIGS. 17(A) and (C), the knitted body element 92 is formed such that a warp thread 92A and a weft thread 92B are knitted so as to intersect each other while extending parallel to the length direction C and the width direction D of the knitted body 90, respectively. Thus, the knitted body element 92 has very little stretchability in the length direction C and the width direction D of the knitted body 90.

When the knitted body element 92 is integrally knitted with the knitted body element 91, the stretchability of the knitted body 90 in the length direction C and the width direction D is limited by the knitted body element 92.

Note that the knitted body element may be constituted by combining at least one of the linear member made of the titanium-tantalum alloy, the linear member made of the polymer, the linear member made of the biodegradable material, and the linear member made of the non-integrated biomaterial. Examples of the combination may include a combination in which the linear member made of the titanium-tantalum alloy is used as the warp thread and at least one of the linear member made of the polymer, the linear member made of the biodegradable material, and the linear member made of the non-integrated biomaterial is used as the weft thread. Further, the combination other than this combination is also included in the present invention as long as the linear member made of the titanium-tantalum alloy is used at least in a part of the warp thread or the weft thread.

Next, referring to FIG. 18, a medical member 9B in a tenth embodiment of the present invention will be described. The medical member 9B is constituted by a knitted body 93. As shown in FIG. 18(A), the knitted body 93 is constituted by two knitted body elements 91 and 94. The knitted body element 91 has already been described in the description of the knitted body 90, and thus a further explanation thereof is omitted herein.

As shown in FIGS. 18(A) and (C), the knitted body element 94 is knitted such that a warp thread 94A extends toward a width direction F of the knitted body 93 while meandering. Thus, the knitted body element 94, which is stretchable in the width direction F by the amount of loosening caused by the meandering of the warp thread 94A, functions like a spring. Thus, the knitted body element 94 is stretchable like a spring in the width direction F of the knitted body 93. Further, a weft thread 94B extends parallel to a length direction E of the knitted body 93 and is knitted so as to connect one end portion of the warp thread 94A to another end portion of the adjacent warp thread 94A.

When the knitted body element having low stretchability is integrally knitted with the knitted body element having high stretchability, the stretchability of the knitted body element having high stretchability is limited by the knitted body element having low stretchability. In the medical member 9B in which the knitted body element 94 is integrally knitted with the knitted body element 91, the stretchability of the knitted body element 94 in the width direction F is limited by the knitted body element 91. Such a knitted body whose stretchability is adjusted by combining the knitted body elements having different stretchability is also included in the present invention. Further, if a weft thread that extends in the length direction E of the knitted body 93 while meandering (not illustrated) is integrally knitted with the knitted body element 91, the stretchability of the weft thread in the length direction E is limited by the knitted body element 91.

Further, the knitted body knitted by any appropriate combination of the knitted body elements 91, 92, and 94 in the knitted bodies 90 and 93 is also included in the present invention.

As described above, when the knitted body elements having different stretching rates in the length direction and the width direction of the knitted body are combined, the stretching rate of one knitted body element can be limited by another knitted body element. In the aforementioned description, the knitted body is constituted by combining two knitted body elements; however, the present invention is not limited thereto, and the knitted body may be constituted by combining three or more knitted body elements. Further, the knitted body may be constituted by a portion constituted by a plurality of the knitted body elements and a portion constituted by combining the single knitted body element.

The density of the mesh, the direction of the mesh, and the like of the knitted body are preferably changed depending on a site where the knitted body is used. For example, in a site where bending and stretching of the soft tissue are desired to be limited, the knitted body knitted so as to have a lower stretching rate is preferably used. On the other hand, in a site where bending and stretching of the soft tissue are tolerated, the knitted body knitted so as to have a higher stretching rate is preferably used. In this manner, the knitted body is easily deformable by following the bending and stretching of the soft tissue.

### 9. Other Embodiments

In the description above, the explanation has been given with the assumption that the net-shaped body 10 and the like are constituted by the knitted body; however, the net-shaped body of the present invention is not limited thereto. For example, the net-shaped body may be obtained by forming a plurality of holes in the sheet body. The hole is formed, for example, by a punch device.

The net-shaped body described above can be used to form a product similar to the net-shaped cylindrical body 11 or the net-shaped sheet body 31. The net-shaped cylindrical body or the net-shaped sheet body formed by the net-shaped body described above is also included in the scope of the present invention. Further, the net-shaped cylindrical body or the net-shaped sheet body formed by the net-shaped body described above can be used according to the use methods described in the first embodiment and the second embodiment.

Further, a material of the net-shaped body described above is similar to that of the net-shaped body 10 described in 1-3. Material of net-shaped body and the description in 1-3. Material of net-shaped body can be applied to the net-shaped body described above.

Further, the medical member formed by appropriately combining the elements described in the above embodiments of the present invention is also included in the present invention.

It is to be understood that the medical member of the present invention is not limited to the above-described embodiments and that various modifications can be made without departing from the scope of the present invention.

1.-27. (canceled)

28. A medical member comprising a knitted body knitted with a linear member formed in a linear shape, wherein
the knitted body is formed in a sheet shape by plain weaving, twilled weaving, plain dutch weaving, twilled dutch weaving, or stockinette stitch, and
at least a part of the knitted body is constituted of an alloy containing titanium, tantalum, and tin, and
the alloy is constituted of 15 at % to 27 at % of tantalum and 1 at % to 8 at % of tin, where the total is 100 at %, with remainder being titanium and unavoidable impurities.

29. The medical member according to claim 28, wherein
the knitted body is constituted by a plurality of knitted body elements, and
the knitted body elements are knitted with the linear members in respective different modes and thus have respective different stretching rates, the stretching rate being represented by a strain per unit tensile force in a tensile direction of the tensile force,
the knitted body is knitted such that the stretching rate of a predetermined one of the knitted body elements is limited by at least one of the remaining knitted body elements knitted into the predetermined knitted body element, and is configured to be attachable to a soft tissue failure site, the soft tissue failure site being a torn site, a damage site, or a defect site of the soft tissue.

30. The medical member according to claim 28, wherein
the knitted body includes:
a first region portion configured to cover a treating region where a soft tissue failure site is treated, the soft tissue failure site being a torn site, a damage site, or a defect site of the soft tissue; and
a second region portion configured to cover a peripheral region of the treating region and be allowed to engage with the soft tissue.

31. The medical member according to claim 30, comprising an engaging member configured to engage the knitted body with the soft tissue.

32. The medical member according to claim 31, wherein the engaging member is a thread member capable of stitching the soft tissue and the knitted body together.

33. The medical member according to claim 30, wherein
the knitted body is constituted by
a net-shaped main body, and
a passage penetrating the main body.

34. The medical member according to claim 33, wherein the knitted body is a stent.

35. The medical member according to claim 28, wherein the knitted body is an embolization member configured to block a blood flow in a blood vessel.

36. The medical member according to claim 28, wherein the knitted body has stretchability.

37. The medical member according to claim 36, wherein the knitted body has a stretching rate of 120% or more, the stretching rate being represented by a strain per unit tensile force in a tensile direction of the tensile force.

38. The medical member according to claim 28, comprising a non-integrated surface that is formed of a non-integrated biomaterial having a property of not being integrated with a biotissue.

39. The medical member according to claim 38, wherein a surface of the knitted body is disposed on a surface opposite to the non-integrated surface.

40. The medical member according to claim 38, wherein the non-integrated biomaterial is a polymer containing at least one component of fluoroethylene, fluoropropylene, and fluoro-polyethylene glycol, a non-crosslinkable silicone polymer, or 2-methacryloyloxyethyl phosphorylcholine.

41. The medical member according to claim 28, comprising a non-integrated biomaterial having a property of not being integrated with a biotissue, and
the non-integrated biomaterial is disposed on the knitted body to form a layer.

42. The medical member according to claim 41, wherein the non-integrated biomaterial is a polymer containing at least one component of fluoroethylene, fluoropropylene, and fluoro-polyethylene glycol, a non-crosslinkable silicone polymer, or 2-methacryloyloxyethyl phosphorylcholine.

43. The medical member according to claim 28, comprising a polymer portion formed of a polymer.

44. The medical member according to claim 43, wherein the polymer portion is formed as a layer of the polymer configured to coat a surface of the knitted body.

45. The medical member according to claim 43, wherein the polymer portion is formed in a net shape or a sheet shape and consecutively connected to the knitted body.

46. The medical member according to claim 28, comprising a layer of hydroxyapatite configured to coat the knitted body.

47. A method for treating a soft tissue using the medical member according to any one of claim 28, comprising:
attaching the knitted body to a soft tissue failure site.
